(19) United States
(12) Patent Application Publication
(10) Pub. No.: US 2023/0414634 A1
(43) Pub. Date: Dec. 28, 2023

(54) METHODS FOR TREATING CHOLESTATIC PRURITUS

(71) Applicant: GlaxoSmithKline Intellectual Property (No.2) Limited, Brentford, Middlesex (GB)

(72) Inventors: Stuart Frederick William KENDRICK, Stevenage (GB); Megan M. MCLAUGHLIN, Collegeville, PA (US)

(21) Appl. No.: 18/031,899

(22) PCT Filed: Oct. 18, 2021
(86) PCT No.: PCT/EP2021/078734

§ 371 Date: Apr. 14, 2023

Related U.S. Application Data
(60) Provisional application No. 63/094,027, filed on Oct. 20, 2020.
(60) Provisional application No. 63/094,426, filed on Oct. 21, 2020.
(60) Provisional application No. 63/144,688, filed on Feb. 2, 2021.

(51) Int. Cl. A61K 31/554; A61P 17/04

(52) U.S. Cl. CPC A61K 31/554; A61P 17/04

(57) ABSTRACT

Methods of treating pruritus, including cholestatic pruritus, particularly cholestatic pruritus in primary biliary cholangitis comprising administration of the compound or a pharmaceutically acceptable salt thereof, are disclosed.

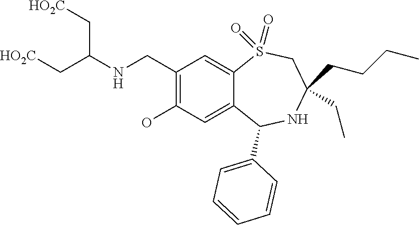 [SMILES: CCCC[C@]1(CC)CS(=O)(=O)c2cc(CNC(CC(=O)O)CC(=O)O)c(O)cc2[C@@H](c2ccccc2)N1]

### FIELD OF THE INVENTION

The present invention relates to methods for treating pruritus, including cholestatic pruritus, particularly cholestatic pruritus in primary biliary cholangitis.

### BACKGROUND OF THE INVENTION

Patent publication WO 2011/137135 discloses, among other compounds, the following ileal bile acid transporter (IBAT) inhibitor compound. This patent publication also discloses methods of synthesis of the compound.

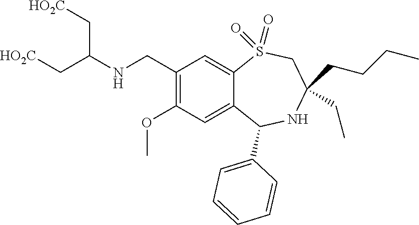 [SMILES: CCCC[C@]1(CC)CS(=O)(=O)c2cc(CNC(CC(=O)O)CC(=O)O)c(OC)cc2[C@@H](c2ccccc2)N1]

The preparation of the above compound is also disclosed in J. Med. Chem, Vol 56, pp 5094-5114 (2013), in J. Org. Chem., Vol 78, pp 12726-12734 (2013) and in patent publications WO 2016/020785 and WO 2018/002827. This compound is also known as linerixibat, GSK2330672 and sometimes abbreviated as GSK672. This compound is in clinical trials for the treatment of cholestatic pruritus in Primary Biliary Cholangitis (PBC).

### SUMMARY OF THE INVENTION

In a first aspect, the present invention provides a method of treatment of cholestatic pruritus in primary biliary cholangitis in a human in need thereof comprising administering to said human the compound

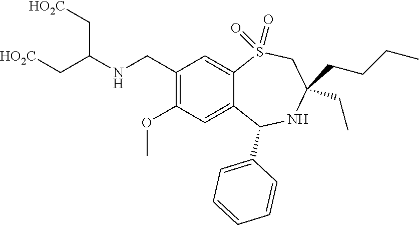 [SMILES: CCCC[C@]1(CC)CS(=O)(=O)c2cc(CNC(CC(=O)O)CC(=O)O)c(OC)cc2[C@@H](c2ccccc2)N1]

or a pharmaceutically acceptable salt thereof, wherein the compound is administered in an amount of 40 mg, twice daily.

In a second aspect, the present invention provides a method of treatment of cholestatic pruritus in primary biliary cholangitis in a human in need thereof comprising administering to said human the compound

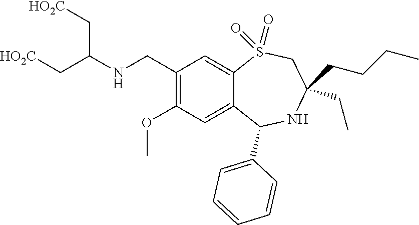 [SMILES: CCCC[C@]1(CC)CS(=O)(=O)c2cc(CNC(CC(=O)O)CC(=O)O)c(OC)cc2[C@@H](c2ccccc2)N1]

or a pharmaceutically acceptable salt thereof, wherein the compound is administered twice daily, and wherein such administration results in improvement in itch.

In a third aspect, the present invention provides the compound

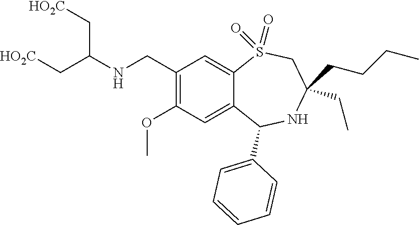 [SMILES: CCCC[C@]1(CC)CS(=O)(=O)c2cc(CNC(CC(=O)O)CC(=O)O)c(OC)cc2[C@@H](c2ccccc2)N1]

or a pharmaceutically acceptable salt thereof, for use in in the treatment of cholestatic pruritus in primary biliary cholangitis wherein the compound is administered in an amount of 40 mg, twice daily.

In a fourth aspect, the present invention provides the compound

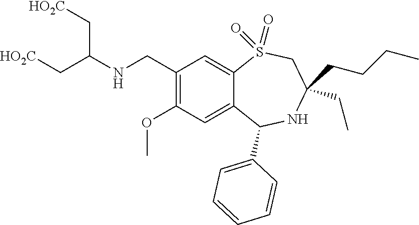 [SMILES: CCCC[C@]1(CC)CS(=O)(=O)c2cc(CNC(CC(=O)O)CC(=O)O)c(OC)cc2[C@@H](c2ccccc2)N1]

or a pharmaceutically acceptable salt thereof, for use in the treatment of cholestatic pruritus in primary biliary cholangitis, wherein the compound is administered twice daily, and wherein such administration results in improvement in itch.

### BRIEF DESCRIPTION OF THE DRAWINGS

FIG. 1(i) shows the clinical study design for the phase 2b clinical trial study GLIMMER NCT02966834

FIG. 1(ii) shows the baseline characteristics of the study participants

FIG. 2 shows the mean serum concentration of biomarker C4 (7-α-hydroxy-4-cholesten-3-one) over time (Intention to Treat (ITT) population)

FIG. 3(i) shows the weekly mean change from baseline in mean Worst Daily Itch Score (A) and change from baseline in Monthly Itch Score over the entire 12-week treatment period (B)

FIG. 3(ii) shows the change from baseline in mean Worst Daily Itch Score at Week 16 (i.e. 4 weeks of placebo followed by 12 weeks of treatment)

FIG. 4 shows the mean difference from placebo in percentage Itch Responder Days during 12 weeks' treatment

FIG. 5a shows the change from baseline in mean sleep score

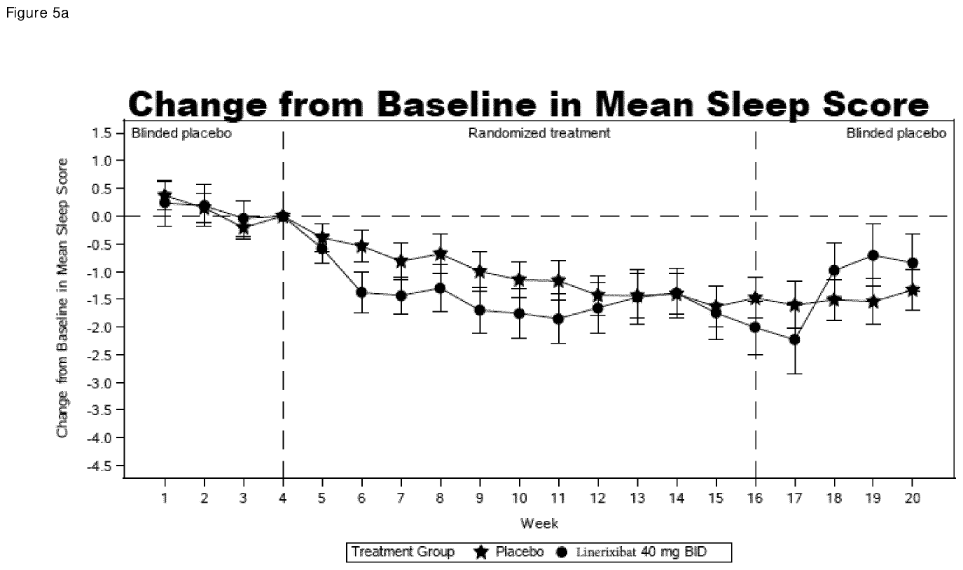

FIG. 5b shows the change from baseline in Worst Daily Itch Score

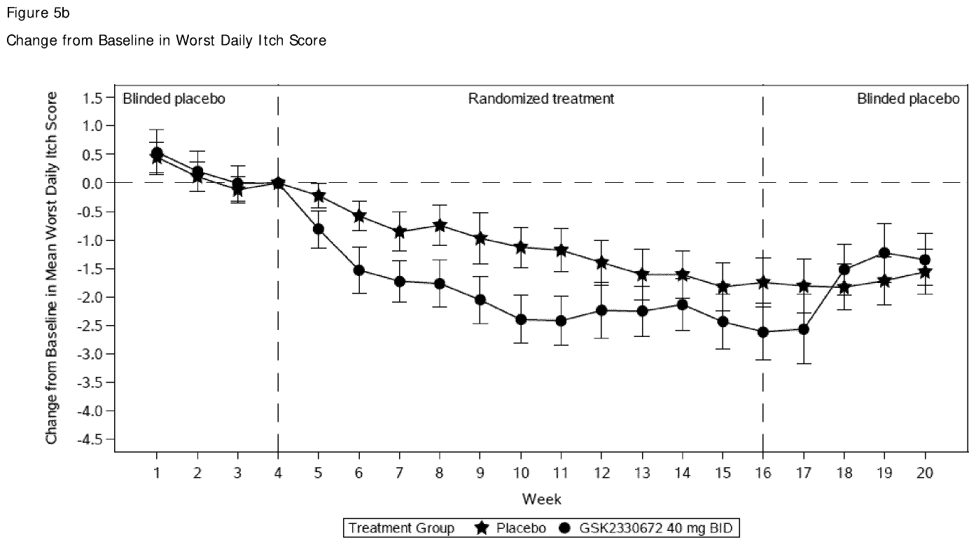

FIG. 6 shows a Bland-Altman graph of the correlation of change from baseline between Monthly Sleep Score and Monthly Itch Score

FIG. 7 shows a graph of biomarker serum fibroblast growth factor-19 (FGF-19) over time (ITT population)

FIG. 8 shows a graph of biomarker serum autotaxin in serum over time (ITT population)

FIG. 9 shows a graph of biomarker serum LDL cholesterol over time (ITT population)

FIG. 10 shows a table of a mixed model repeated measures (MMRM) analysis of geometric least squares mean ratio of total serum bile acids (TSBA) and C4 (ITT population; post hoc analysis)

### DETAILED DESCRIPTION OF THE INVENTION

Cholestatic pruritus affects up to 75% of patients with PBC and significantly impacts quality of life (QOL). Recently, novel pharmacologic treatments for PBC have been developed. Ursodeoxycholic acid (UDCA) and obeticholic acid (OCA) are currently approved or available for clinical use; however, they do not ameliorate cholestatic pruritus (Levy C. Management of pruritus in patients with cholestatic liver disease. Gastroenterol Hepatol (N Y) 2011;7:615-7; Santiago P, Scheinberg A R, Levy C. Cholestatic liver diseases: new targets, new therapies. Therap Adv Gastroenterol 2018;11:1756284818787400). In fact, OCA has a labelled warning and precaution for pruritus, potentially limiting its use in clinical practice.

Bile acids have been identified as potential targets for therapeutic intervention based on the hypothesis that, in cholestasis, bile acids accumulate in liver, serum, and skin, contributing to pruritus. Furthermore, bile acid binding resins, such as cholestyramine, are recommended as first-line therapy for pruritus in PBC (Hegade VS, Bolier R, Oude Elferink RP, et al. A systematic approach to the management of cholestatic pruritus in primary biliary cirrhosis. Frontline Gastroenterol 2016;7:158-166). However, cholestyramine does not provide sufficient relief of pruritus for all patients, and adherence can be low due to its unpalatable taste and gastrointestinal-associated adverse events (AEs).

Various other systemic diseases are associated with chronic pruritus. Up to 20% of patients with generalized pruritus suffer from systemic disease entities such as chronic kidney disease, hepatobiliary disorder, and hematological disorder. Almost all hepatobiliary disorders may be accompanied by pruritus although this symptom is commonly seen in disorders with cholestatic features such as PBC. (Dull M M, Kremer A E. Newer Approaches to the Management of Pruritus in Cholestatic Liver Disease. Curr Hepatol Rep 2020;19:86-95; Hegade V S, Kendrick S F, Jones D E. Drug treatment of pruritus in liver diseases. Clin Med (Lond). 2015;15(4):351-357; World J Gastroenterol. May 21, 2017; 23(19): 3418-3426. Recent advances in the management of pruritus in chronic liver diseases).

Off-label therapies such as cholestyramine, rifampin, bezafibrate, selective opioid receptor antagonist agents (such as naloxone and naltrexone), and sertraline are second-line treatment options for pruritus in liver disease. Bezafibrate showed a reduction in pruritus compared with placebo (Corpechot C, Chazouillères O, Rousseau A, et al. A Placebo-Controlled Trial of Bezafibrate in Primary Biliary Cholangitis. N Engl J Med 2018;378:2171-2181); however, fibrates can be contraindicated in hepatic impairment. Nalfurafine has also demonstrated small but significant improvements in itch in a patient population with a variety of liver diseases. (Yagi M, Tanaka A, Namisaki T, et al. Is patient-reported outcome improved by nalfurafine hydrochloride in patients with primary biliary cholangitis and refractory pruritus? A post-marketing, single-arm, prospective study. J Gastroenterol 2018;53:1151-1158). Despite availability of these recommended therapies for pruritus, patients remain significantly symptomatic; real-world data from 2194 patients in the UK-PBC cohort study found over one-third of patients experienced persistent pruritus since diagnosis and almost three-quarters experienced pruritus during their disease course; severity of pruritus persisted over a 6-year period.

BYLVAY (odevixibat), has recently been approved by the FDA for the treatment of pruritus in progressive familial intrahepatic cholestasis (PFIC). Odevixibat is an IBAT inhibitor compound and it is administered once daily for this indication.

LIVMARLI (maralixibat) oral solution has recently been approved by the FDA for treating cholestatic pruritus in children who have Alagille syndrome (ALGS). Maralixibat is an IBAT inhibitor compound and it is administered once daily for this indication.

This underlines the continuing unmet clinical need for more effective treatment options for patients with pruritus, including cholestatic pruritus, particularly cholestatic pruritus in primary biliary cholangitis.

Pruritus is a patient reported outcome and cannot be directly measured. Rather, it may be estimated by various instruments. Cholestatic pruritus can be measured using different rating scales such as:

i) 10 point Numerical Rating Scale (NRS): “Development and adaptation of patient-reported outcome measures for patients who experience itch associated with primary biliary cholangitis.” J Patient Rep Outcomes. 2019;3(1):2

ii) 5D Itch Scale: Elman et al., “The 5-D itch scale: a new measure of pruritus.” Br J Dermatol. 2010 Mar;162(3):587-93

iii) PBC-40 (PBC-40 HRQOL): Jacoby et al., “Development, validation, and evaluation of the PBC-40, a disease specific health related quality of life measure for primary biliary cirrhosis.” Gut. 2005 Nov;54(11):1622-9. The six domains of PBC-40 relate to fatigue, emotional, social, and cognitive function, general symptoms, and itch.

Measurement of Itch (pruritus)—10 Point Numerical Rating Scale (NRS)
Itch Score: itch scores recorded by study participants twice daily; a 0-10 Numerical Rating Scale (NRS) is used to assess itch with a response option of 0 representing no itching and 10 the worst imaginable itching
Worst Daily Itch Score: the worst of two itch scores recorded by a patient daily is considered the score for that day (i.e. the most severe (highest) NRS recorded on a given day)
Mean Worst Daily Itch Score (Weekly Itch Score [WIS]): the average of the Worst Daily Itch Scores in one week (7 days)
Monthly Itch Score (MIS): the worst Mean Worst Daily Itch Score for that month (i.e., worst week score of the 4 weeks)
Itch Response: for example, ≥2-point improvement in mean Worst Daily Itch Score from Baseline
Itch Responder Days: days when the Itch Response definition is met (i.e. days with an Itch Response)
Itch Severity: measured using 0-10 NRS; moderate to severe is ≥4 NRS
Baseline (itch score at the end of the placebo period before treatment,) is the average of the itch scores in the 7 days prior to randomisation (in Example 2, prior to randomisation at Week 4)
Change from Baseline was calculated as the post-Baseline value minus the Baseline value.

The present invention provides a method of treatment of pruritus in a human in need thereof comprising administering to said human the compound

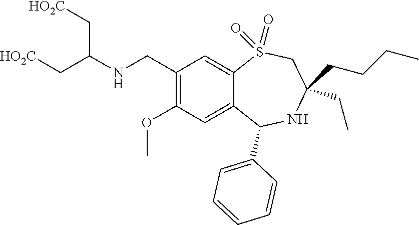 [SMILES: CCCC[C@]1(CC)CS(=O)(=O)c2cc(CNC(CC(=O)O)CC(=O)O)c(OC)cc2[C@@H](c2ccccc2)N1]

or a pharmaceutically acceptable salt thereof, wherein the compound is administered twice daily, and wherein such administration results in improvement in itch.

The present invention also provides the compound

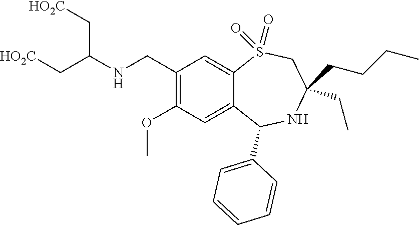 [SMILES: CCCC[C@]1(CC)CS(=O)(=O)c2cc(CNC(CC(=O)O)CC(=O)O)c(OC)cc2[C@@H](c2ccccc2)N1]

or a pharmaceutically acceptable salt thereof, for use in in the treatment of pruritus, wherein the compound is administered twice daily, and wherein such administration results in improvement in itch.

As mentioned hereinabove, various systemic diseases are associated with chronic pruritus. Pruritus is common in liver disease as almost all hepatobiliary disorders may be accompanied by pruritus although this symptom is commonly seen in disorders with cholestatic features such as PBC. Cholestatic pruritus is a debilitating itch as a result of disfunction of the bile ducts of the liver and thought to be caused by release of toxic bile acids throughout the body. This itch can occur in multiple liver diseases and is recognized as a major symptom of PBC.

In one embodiment, the pruritus is associated with a liver disease. In another embodiment, the pruritus is associated with a chronic liver disease, for example a cholestatic liver disease. In a further embodiment, the pruritis is cholestatic pruritus. In a further embodiment, the pruritis is cholestatic pruritus in PBC.

In one embodiment, the present invention provides the compound

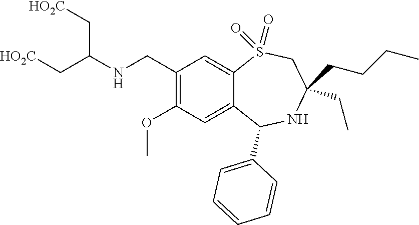 [SMILES: CCCC[C@]1(CC)CS(=O)(=O)c2cc(CNC(CC(=O)O)CC(=O)O)c(OC)cc2[C@@H](c2ccccc2)N1]

or a pharmaceutically acceptable salt thereof, for use in the treatment of cholestatic pruritus in primary biliary cholangitis wherein the compound is administered in an amount of between 3 mg and 100 mg, twice daily orally, and wherein such administration results in improvement in itch which is demonstrated by a mean difference between placebo in average change from baseline of Monthly Itch Score of at least −0.5.

In one embodiment, the present invention provides a method of treatment of cholestatic pruritus in primary biliary cholangitis in a human in need thereof comprising administering to said human the compound

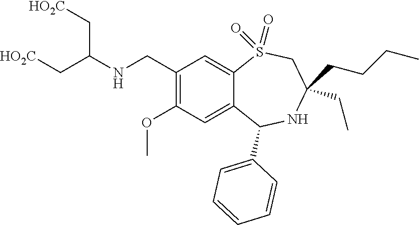 [SMILES: CCCC[C@]1(CC)CS(=O)(=O)c2cc(CNC(CC(=O)O)CC(=O)O)c(OC)cc2[C@@H](c2ccccc2)N1]

wherein the compound is administered twice daily orally, and wherein such administration results in improvement in itch, wherein the improvement in itch is statistically different from placebo as measured by Mean Worst Daily Itch Score.

In one embodiment, the present invention provides the compound

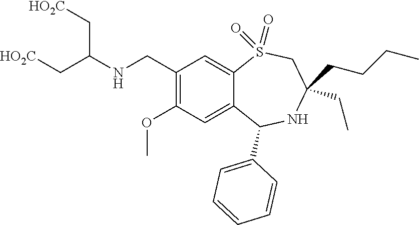 [SMILES: CCCC[C@]1(CC)CS(=O)(=O)c2cc(CNC(CC(=O)O)CC(=O)O)c(OC)cc2[C@@H](c2ccccc2)N1]

or a pharmaceutically acceptable salt thereof, for use in the treatment of cholestatic pruritus in primary biliary cholangitis wherein the compound is administered in an amount of between 3 mg and 100mg, twice daily orally, and wherein such administration results in improvement in itch which is demonstrated by a mean difference from placebo in mean change from baseline Monthly Itch Score of at least −0.5.

In one embodiment, the present invention provides the compound

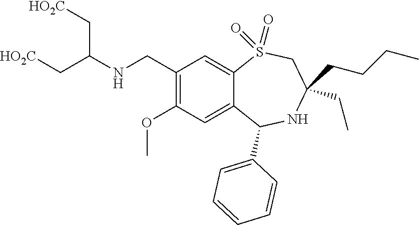 [SMILES: CCCC[C@]1(CC)CS(=O)(=O)c2cc(CNC(CC(=O)O)CC(=O)O)c(OC)cc2[C@@H](c2ccccc2)N1]

or a pharmaceutically acceptable salt thereof, for use in the treatment of cholestatic pruritus in primary biliary cholangitis wherein the compound is administered twice daily orally, and wherein such administration results in improvement in itch, wherein the improvement in itch is statistically different from placebo as measured by Mean Worst Daily Itch Score.

In one embodiment, the present invention provides a method of treatment of cholestatic pruritus in primary biliary cholangitis in a human in need thereof, wherein the human has an NRS of ≥4 on the 10 point NRS, comprising administering to said human the compound

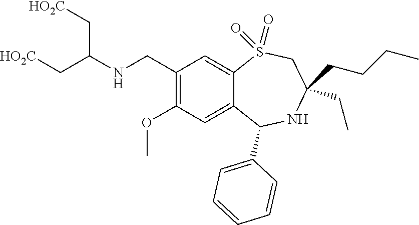 [SMILES: CCCC[C@]1(CC)CS(=O)(=O)c2cc(CNC(CC(=O)O)CC(=O)O)c(OC)cc2[C@@H](c2ccccc2)N1]

or a pharmaceutically acceptable salt thereof, wherein the compound is administered twice daily.

In one embodiment, the present invention provides the compound

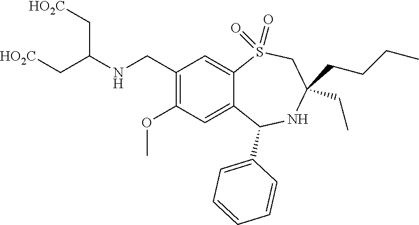 [SMILES: CCCC[C@]1(CC)CS(=O)(=O)c2cc(CNC(CC(=O)O)CC(=O)O)c(OC)cc2[C@@H](c2ccccc2)N1]

or a pharmaceutically acceptable salt thereof, for use in the treatment of cholestatic pruritus in primary biliary cholangitis in a patient which has an NRS of ≥4 on the 10 point NRS, wherein the compound is administered twice daily.

In one embodiment, in respect of the methods of treatment or the compound for use in treatment as described herein, the compound is administered orally.

In respect of the methods of treatment or the compound for use in treatment as described herein:

In one embodiment, the compound is administered in an amount of between 3 mg and 100 mg, twice daily, for example between 30 mg and 100 mg twice daily.

In one embodiment, the compound is administered in an amount of between 40 mg and twice daily.

In one embodiment, the compound is administered in an amount of approximately twice daily.

In one embodiment, the compound is administered in an amount of approximately twice daily.

In a comparative example, the compound is administered in an amount of approximately 180 mg, once daily.

In a comparative example, the compound is administered in an amount of approximately once daily.

In respect of the methods of treatment or the compound for use in treatment as described herein: In one embodiment, an improvement in itch refers to the situation where the subject's Mean Worst Daily Itch Score on the 10 point Numerical Rating Scale is improved at the end of the treatment period relative to baseline. In one particular embodiment, the treatment period is at least 12 weeks in duration. In another embodiment, the treatment period is at least 24 weeks in duration.

In one embodiment, said improvement in itch is demonstrated by a mean difference from placebo in mean change from baseline of Monthly Itch Score. In another embodiment, said improvement in itch is demonstrated by a mean difference from placebo in mean change from baseline of Monthly Itch Score of at least −0.1, or at least −0.2 or at least −0.3, or at least −0.4. In another embodiment, said improvement in itch is demonstrated by a mean difference from placebo in mean change from baseline of Monthly Itch Score of at least −0.5. In another embodiment, said improvement in itch is demonstrated by a mean difference from placebo in mean change from baseline of Monthly Itch Score of at least −0.6, or at least −0.7 or at least −0.8. In another embodiment, said improvement in itch is demonstrated by a mean difference from placebo in mean change from baseline of Monthly Itch Score of at approximately −0.9.

In one embodiment, said improvement in itch is demonstrated by a change from baseline Monthly Itch Score of at least −0.5, for example at least −0.9, or alternatively approximately −0.9. In one embodiment, said improvement in itch is demonstrated by a change from baseline Monthly Itch Score of approximately −0.9 over 24 weeks.

In one embodiment, said improvement in itch is statistically different from placebo as measured by Mean Worst Daily Itch Score.

In one embodiment, the compound administered according to the methods of treatment, or the compound for use in treatment as described herein is the compound:

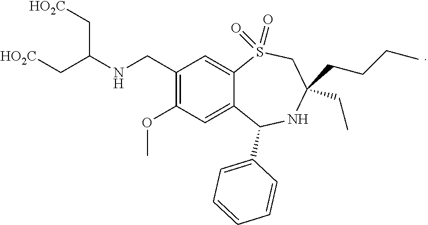 [SMILES: CCCC[C@]1(CC)CS(=O)(=O)c2cc(CNC(CC(=O)O)CC(=O)O)c(OC)cc2[C@@H](c2ccccc2)N1]

In respect of the methods of treatment or the compound for use in treatment as described herein: in one embodiment, the compound or a pharmaceutically acceptable salt thereof is formulated into a tablet. In one embodiment, the tablet further comprises filler, disintegrant, and lubricant. In one embodiment the tablet comprises from 10 to 100 mg of the compound or a pharmaceutically acceptable salt thereof. In one embodiment, the tablet comprises from 40 to 90 mg of the compound or a pharmaceutically acceptable salt thereof. In another embodiment, the tablet comprises 90 mg of the compound or a pharmaceutically acceptable salt thereof. In another embodiment, the tablet comprises 45 mg of the compound or a pharmaceutically acceptable salt thereof. In another embodiment, the tablet comprises 40 mg of the compound or a pharmaceutically acceptable salt thereof. One example of a suitable tablet is a tablet comprising the compound, microcrystalline cellulose, and magnesium stearate. Another example of a suitable tablet is a tablet comprising the compound, microcrystalline cellulose, magnesium stearate and croscarmellose sodium.

### Pharmacodynamic Biomarkers

The following biomarkers are useful in clinical investigations relating to the treatment of pruritus, particularly cholestatic pruritus:

Hegade et al, Liver International. 2019;39:967-975

Hegade et al, Lancet 389 March 18, 2017; 1114-1123

Serum 7-α-hydroxy-4-cholesten-3-one (C4)

C4 is an intermediate in the biochemical synthesis of bile acids from cholesterol. C4 concentrations reflect the activity of the bile acid synthetic pathway.

### Serum Fibroblast Growth Factor-19 (FGF-19)

FGF-19 a protein which functions as a hormone, regulating bile acid synthesis, with effects on glucose and lipid metabolism.

### Serum Autotaxin (ATX)

Autotaxin is the enzyme that produces lysophosphatidic acid (LPA) which is found to correlate with itch intensity.

### Serum Low-Density Lipoprotein Cholesterol (LDL)

LDL one of the five major groups of lipoprotein which transport all fat molecules around the body in the extracellular water.

### Total Serum Bile Acids (TSBA)

TSBA are metabolized in the liver and can serve as a marker for normal liver function.

The present invention also provides a method of treatment of cholestatic pruritus in primary biliary cholangitis in a human in need thereof comprising administering to said human the compound

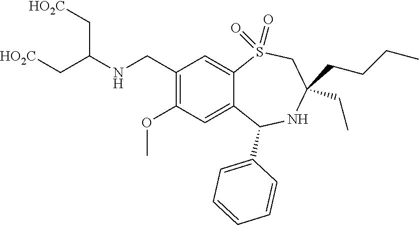 [SMILES: CCCC[C@]1(CC)CS(=O)(=O)c2cc(CNC(CC(=O)O)CC(=O)O)c(OC)cc2[C@@H](c2ccccc2)N1]

or a pharmaceutically acceptable salt thereof, wherein the compound is administered twice daily, and wherein such administration results in a decrease of total serum bile acids in the human, or in a population of patients.

The present invention also provides the compound

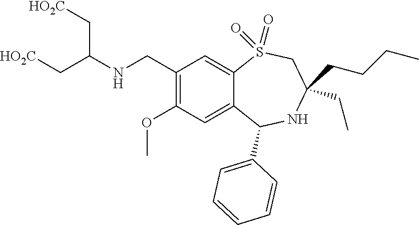 [SMILES: CCCC[C@]1(CC)CS(=O)(=O)c2cc(CNC(CC(=O)O)CC(=O)O)c(OC)cc2[C@@H](c2ccccc2)N1]

or a pharmaceutically acceptable salt thereof, for use in the treatment of cholestatic pruritus in primary biliary cholangitis, wherein the compound is administered twice daily, and wherein such administration results in a decrease of total serum bile acids in the human, or in a population of patients.

In one embodiment, a decrease of total serum bile acids is achieved when the level of total serum bile acids in the human is lower at the end of the treatment period compared to the beginning of the treatment period (baseline).

In another embodiment, a decrease of total serum bile acids is achieved when the mean level of total serum bile acids in a population of patients is lower at the end of the treatment compared to the beginning of the treatment period (baseline). In another embodiment, a decrease of total serum bile acids is achieved when the mean level of total serum bile acids in a population of patients is lower at the end of the treatment compared to the beginning of the treatment period (baseline), as compared to placebo.

In one embodiment, the mean decrease of total serum bile acids is a statistically significant mean change from baseline, compared to placebo. In another embodiment, the decrease of total serum bile acids is at least 10%, 20%, 30%, 40%, 50%, 60%, 70%, 80% or 100%.

In one embodiment, the treatment period is at least 12 weeks.

In one embodiment, a population of patients is at least 30 patients, or at least 50 patients, or at least 100 patients.

The present invention also provides a method of treatment of cholestatic pruritus in primary biliary cholangitis in a human in need thereof comprising administering to said human the compound

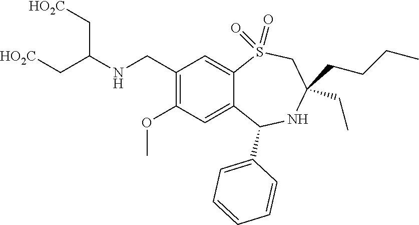 [SMILES: CCCC[C@]1(CC)CS(=O)(=O)c2cc(CNC(CC(=O)O)CC(=O)O)c(OC)cc2[C@@H](c2ccccc2)N1]

or a pharmaceutically acceptable salt thereof, wherein the compound is administered twice daily, and wherein such administration results in an increase of serum C4 in the human, or in a population of patients.

The present invention also provides the compound

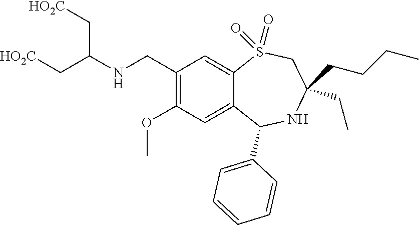 [SMILES: CCCC[C@]1(CC)CS(=O)(=O)c2cc(CNC(CC(=O)O)CC(=O)O)c(OC)cc2[C@@H](c2ccccc2)N1]

or a pharmaceutically acceptable salt thereof, for use in the treatment of cholestatic pruritus in primary biliary cholangitis, wherein the compound is administered twice daily, and wherein such administration results in an increase of serum C4 in the human, or in a population of patients.

In one embodiment, an increase of serum C4 is achieved when the level of serum C4 in the human is higher at the end of the treatment period compared to the beginning of the treatment period (baseline).

In another embodiment, an increase of serum C4 is achieved when the mean level of serum C4 in a population of patients is higher at the end of the treatment compared to the beginning of the treatment period (baseline). In another embodiment, an increase of serum C4 is achieved when the mean level of serum C4 in a population of patients is higher at the end of the treatment compared to the beginning of the treatment period (baseline), as compared to placebo.

In one embodiment, the mean increase of serum C4 is a statistically significant mean change from baseline, compared to placebo. In another embodiment, the increase of serum C4 is at least 10%, 20%, 30%, 40%, 50%, 60%, 70%, 80% or 100%.

In one embodiment, the treatment period is at least 12 weeks.

In one embodiment, a population of patients is at least 30 patients, or at least 50 patients, or at least 100 patients.

The present invention also provides a method of treatment of cholestatic pruritus in primary biliary cholangitis in a human in need thereof comprising administering to said human the compound

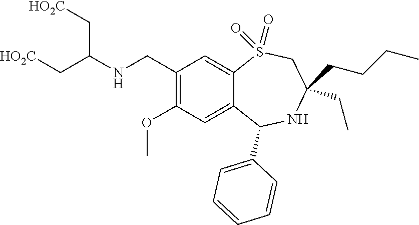 [SMILES: CCCC[C@]1(CC)CS(=O)(=O)c2cc(CNC(CC(=O)O)CC(=O)O)c(OC)cc2[C@@H](c2ccccc2)N1]

or a pharmaceutically acceptable salt thereof, wherein the compound is administered twice daily, and wherein such administration results in a mean decrease of serum fibroblast growth factor 19 in the human, or in a population of patients.

The present invention also provides the compound

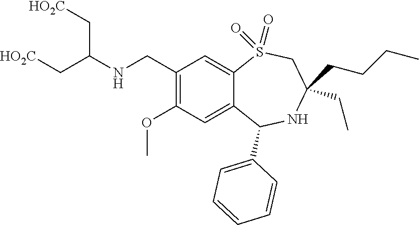 [SMILES: CCCC[C@]1(CC)CS(=O)(=O)c2cc(CNC(CC(=O)O)CC(=O)O)c(OC)cc2[C@@H](c2ccccc2)N1]

or a pharmaceutically acceptable salt thereof, for use in the treatment of cholestatic pruritus in primary biliary cholangitis, wherein the compound is administered twice daily, and wherein such administration results in a mean decrease of serum fibroblast growth factor 19 in the human, or in a population of patients.

In one embodiment, a decrease of serum fibroblast growth factor 19 is achieved when the level of serum fibroblast growth factor 19 in the human is lower at the end of the treatment period compared to the beginning of the treatment period (baseline).

In another embodiment, a decrease of serum fibroblast growth factor 19 is achieved when the mean level of serum fibroblast growth factor 19 in a population of patients is lower at the end of the treatment compared to the beginning of the treatment period (baseline). In another embodiment, a decrease of serum fibroblast growth factor 19 is achieved when the mean level of serum fibroblast growth factor 19 in a population of patients is lower at the end of the treatment compared to the beginning of the treatment period (baseline), as compared to placebo.

In one embodiment, the mean decrease of serum fibroblast growth factor 19 is a statistically significant mean change from baseline, compared to placebo. In another embodiment, the decrease of serum fibroblast growth factor 19 is at least 10%, 20%, 30%, 40%, 50%, 60%, 70%, 80% or 100%.

In one embodiment, the treatment period is at least 12 weeks.

In one embodiment, a population of patients is at least 30 patients, or at least 50 patients, or at least 100 patients.

The present invention also provides a method of treatment of cholestatic pruritus in primary biliary cholangitis in a human in need thereof comprising administering to said human the compound

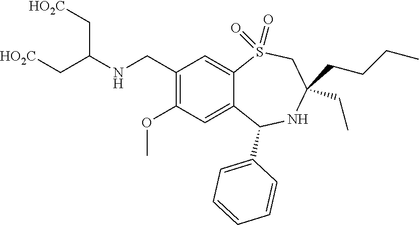 [SMILES: CCCC[C@]1(CC)CS(=O)(=O)c2cc(CNC(CC(=O)O)CC(=O)O)c(OC)cc2[C@@H](c2ccccc2)N1]

or a pharmaceutically acceptable salt thereof, wherein the compound is administered twice daily, and wherein such administration results in a mean decrease of serum autotaxin in the human.

The present invention also provides the compound

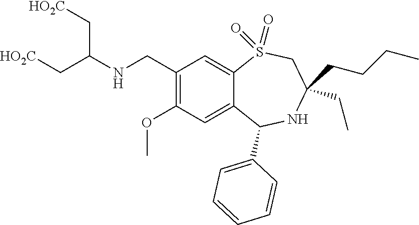 [SMILES: CCCC[C@]1(CC)CS(=O)(=O)c2cc(CNC(CC(=O)O)CC(=O)O)c(OC)cc2[C@@H](c2ccccc2)N1]

or a pharmaceutically acceptable salt thereof, for use in the treatment of cholestatic pruritus in primary biliary cholangitis, wherein the compound is administered twice daily, and wherein such administration results in a mean decrease of serum autotaxin in the human.

In one embodiment, a decrease of serum autotaxin is achieved when the level of serum autotaxin in the human is lower at the end of the treatment period compared to the beginning of the treatment period (baseline).

In another embodiment, a decrease of serum autotaxin is achieved when the mean level of serum autotaxin in a population of patients is lower at the end of the treatment compared to the beginning of the treatment period (baseline). In another embodiment, a decrease of serum autotaxin is achieved when the mean level of serum autotaxin in a population of patients is lower at the end of the treatment compared to the beginning of the treatment period (baseline), as compared to placebo.

In one embodiment, the mean decrease of serum autotaxin is a statistically significant mean change from baseline, compared to placebo. In another embodiment, the decrease of serum autotaxin is at least 10%, 20%, 30%, 40%, 50%, 60%, 70%, 80% or 100%.

In one embodiment, the treatment period is at least 12 weeks.

In one embodiment, a population of patients is at least 30 patients, or at least 50 patients, or at least 100 patients.

In the clinical study Example 2, itch symptoms closely correlated with sleep interference, suggesting the potential for a possible close clinical relationship between itch and sleep. In addition to improving the direct impact of the symptom of itch, an improvement in itch may have an impact on sleep. This is of importance, as sleep disturbance is a common complaint of patients with PBC that adds to disease burden.

Accordingly, the present invention provides a method of treatment of cholestatic pruritus in primary biliary cholangitis in a human in need thereof comprising administering to said human the compound

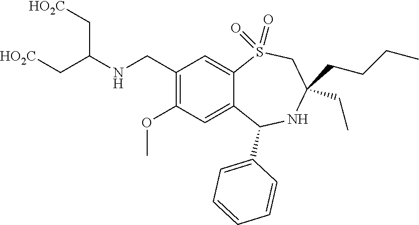 [SMILES: CCCC[C@]1(CC)CS(=O)(=O)c2cc(CNC(CC(=O)O)CC(=O)O)c(OC)cc2[C@@H](c2ccccc2)N1]

or a pharmaceutically acceptable salt thereof, wherein the compound is administered twice daily orally, and wherein such administration results in improvement in itch and/or sleep.

The present invention also provides a method of treatment of cholestatic pruritus in primary biliary cholangitis in a human in need thereof comprising administering to said human the compound

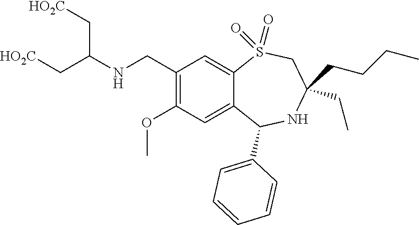 [SMILES: CCCC[C@]1(CC)CS(=O)(=O)c2cc(CNC(CC(=O)O)CC(=O)O)c(OC)cc2[C@@H](c2ccccc2)N1]

or a pharmaceutically acceptable salt thereof, wherein the compound is administered twice daily orally, and wherein such administration results in improvement in itch and sleep.

In a yet further aspect, the present invention provides a compound for use in the treatment of cholestatic pruritus in primary biliary cholangitis in a human in need thereof comprising administering to said human the compound

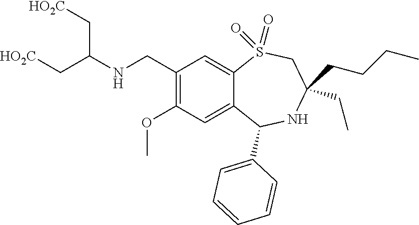 [SMILES: CCCC[C@]1(CC)CS(=O)(=O)c2cc(CNC(CC(=O)O)CC(=O)O)c(OC)cc2[C@@H](c2ccccc2)N1]

or a pharmaceutically acceptable salt thereof, wherein the compound is administered twice daily orally, and wherein such administration results in improvement in itch and/or sleep.

In a yet further aspect, the present invention provides a compound for use in the treatment of cholestatic pruritus in primary biliary cholangitis in a human in need thereof comprising administering to said human the compound

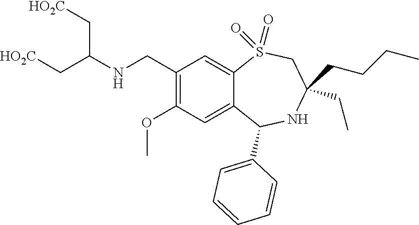 [SMILES: CCCC[C@]1(CC)CS(=O)(=O)c2cc(CNC(CC(=O)O)CC(=O)O)c(OC)cc2[C@@H](c2ccccc2)N1]

or a pharmaceutically acceptable salt thereof, wherein the compound is administered twice daily orally, and wherein such administration results in improvement in itch and sleep.

An improvement in sleep in a patient being treated may be demonstrated by a reduction in sleep interference in said patient. In one embodiment, said improvement in sleep is demonstrated by a change from baseline Monthly Sleep Score.

In one embodiment, an improvement in sleep refers to the situation where the subject's Mean Worst Daily Sleep Score on the 10 point Numerical Rating Scale is improved at the end of the treatment period relative to baseline. In one particular embodiment, the treatment period is at least 12 weeks in duration.

The present invention further provides a method of improving quality of life in a human in need thereof comprising administering to said human the compound

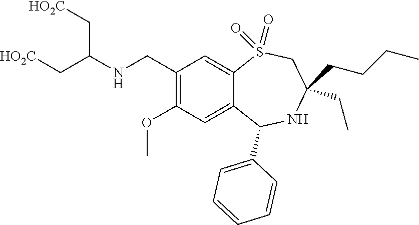 [SMILES: CCCC[C@]1(CC)CS(=O)(=O)c2cc(CNC(CC(=O)O)CC(=O)O)c(OC)cc2[C@@H](c2ccccc2)N1]

or a pharmaceutically acceptable salt thereof, wherein the compound is administered twice daily orally, and wherein such administration results in improvement social and emotional domains as measured by the PBC-40 HRQOL assessment scale.

The present invention further provides the compound

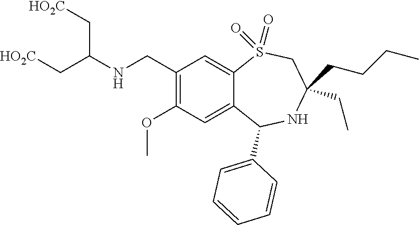 [SMILES: CCCC[C@]1(CC)CS(=O)(=O)c2cc(CNC(CC(=O)O)CC(=O)O)c(OC)cc2[C@@H](c2ccccc2)N1]

or a pharmaceutically acceptable salt thereof, for use in the improving quality of life in a human, wherein the compound is administered twice daily orally, and wherein such administration results in improvement social and emotional domains as measured by the PBC-40 HRQOL assessment scale.

### TREATMENT OF A CHOLESTATIC LIVER DISEASE

### Example 1

A Phase 2a clinical study (NCT01899703; GSK study BAT117213) was carried out to investigate the safety, tolerability, and effect of repeat doses of GSK672 administration in patients with cholestatic pruritus in Primary Biliary Cholangitis (PBC). The results of this study have been summarized and published on clintrials.gov (NCT01899703).

A phase 2 double-blind, randomised, placebo controlled, crossover trial in PBC patients with pruritus was conducted at two specialist PBC centres in the United Kingdom between March 2014 and November 2015. Subjects received oral 45 to 90 mg GSK672 (45 mg BID for Day 1 to 3 and 90 mg BID for Days 4 to 14) and placebo twice daily for 14 days, in a crossover sequence.

The three different itch rating scales were employed: 10-point NRS, 5D Itch Scale and PBC-40.

The primary end point was safety (measured by clinical and laboratory assessments and adverse events (AEs)) and tolerability. Secondary end points were: i) changes in pruritus scores from baseline measured using a 0 to 10 numerical rating scale (NRS) completed twice daily and PBC-40 itch domain scores and 5-D itch scale and ii) changes in serum levels of total bile acids and C4. Serum levels of ATX activity and FGF-19 were measured at baseline and at the end of each treatment period.

An analysis was carried out as to the effect of 90 mg BID. No analysis was carried out as to the effect of 45 mg BID as this dose was administered for Days 1 to 3 in order to ease patients onto the higher 90 mg BID dose. GSK672 90 mg BID showed significant reduction (e.g. 71%) as compared to placebo in itch intensity as measured by NRS [−1.58 (95%CI: −2.48 to −0.68)], PBC-40 itch domain [−0.59 (95% CI: −0.94 to −0.24)] and 5-D itch [−4.55 (95% CI: −6.60 to −2.49)].

In summary, two weeks of oral twice daily 90 mg GSK672 was well tolerated and reduced itch intensity in a high proportion of PBC patients with pruritus. In this randomized placebo controlled 14 day cross-over study in 22 subjects with PBC pruritus (Study BAT117213), GSK672 at 90 mg BID resulted in a statistically significant decrease in pruritus severity compared to placebo as evidenced by the 3 different rating scales NRS, 5D Itch Scale and PBC-40.

Reduction of pruritus severity occurred within the first week of GSK2330672, continued to decrease through 2 weeks of treatment and returned towards baseline upon blinded switch to placebo. Decreases in fatigue, sleep disturbance and overall disability were also noted upon GSK2330672 administration compared to placebo.

### Example 2

GLIMMER trial: Phase 2b study (NCT02966834; GSK study 201000). A randomized, double-blind, placebo-controlled study of linerixibat, an inhibitor of the ileal bile acid transporter, in the treatment of cholestatic pruritus in primary biliary cholangitis, for patients with PBC and pruritus.

### Background

Cholestatic pruritus significantly impairs the quality of life (QOL) of patients with primary biliary cholangitis (PBC). Linerixibat (GSK2330672), a minimally absorbed oral small molecule inhibitor of the human ileal bile acid transporter (IBAT), is being developed to treat cholestatic pruritus in PBC. This trial assessed the dose response and tolerability of linerixibat compared with placebo in patients with cholestatic pruritus with PBC.

### Methods

### Study Design

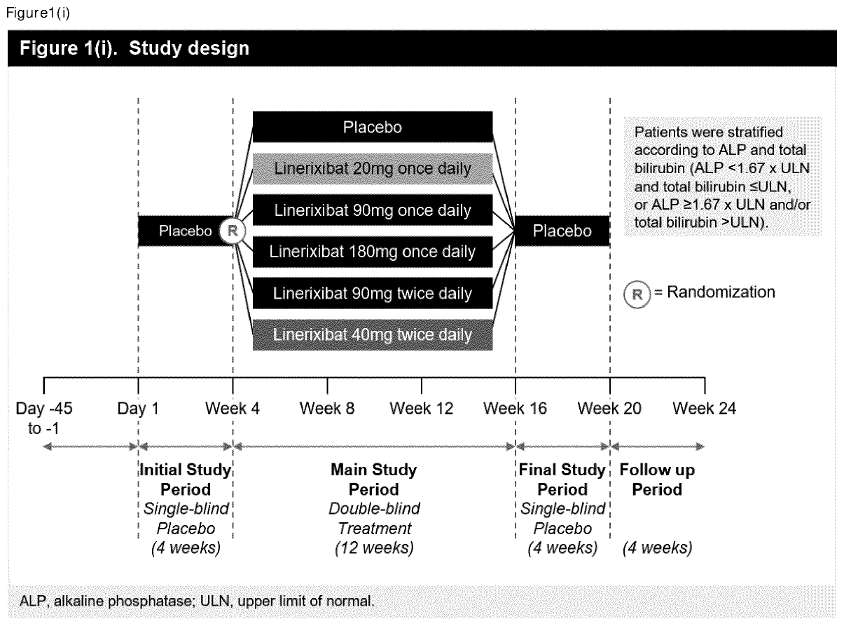

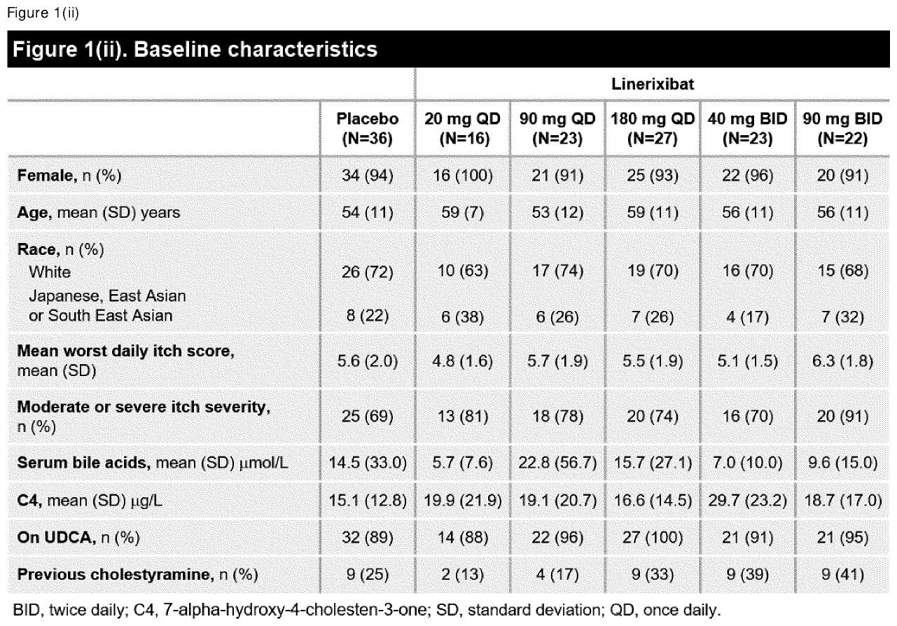

### See FIG. 1(i) for Study Design.

The twice daily patient-reported 0-10 Numerical Rating Scale (NRS) worst itch assessment was employed, with the worst value from each day used to calculate the mean worst daily itch, which was then used to assess change in pruritus. This measure has independent content validity in PBC; similar measures are psychometrically valid and have been used to determine changes in itch intensity in PBC.

Patients with PBC with pruritus (≥4 on 0-10 numeric rating scale [NRS]) were enrolled; double-blind treatment was preceded and followed by 4 weeks' single-blind placebo.

At Week 4, patients with NRS ≥3 were randomized,(4:1) to double-blind linerixibat or placebo for 12 weeks (to Week 16), followed by single-blind placebo (to Week 20).

Stable anti-itch therapy and ursodeoxycholic acid (UDCA) were permitted.

### Endpoints

Primary endpoint: mean change from baseline in mean Worst Daily Itch Score at Week 16. Participants were required to score the severity of their itching using the 0-10 NRS, from which the Worst Daily Itch Score is obtained, and the Mean Worst Daily Itch score was calculated as the average of the worst daily itch scores provided in the 7 days prior to the Week 16 visit. Baseline is the average of the scores in the 7 days prior to the Week 4, and the Change from Baseline was calculated. Analysis was done using Analysis of covariance (ANCOVA) including treatment group and centered Mean Worst Daily Itch score at Baseline.

Secondary and exploratory endpoints: itch efficacy, quality of life (PBC-40) and pharmacodynamic biomarkers, and safety and tolerability.

Ad hoc endpoint: change from baseline itch compared with Monthly Itch Score over the main study period.

### Results

Patients

147 patients were randomized; baseline characteristics are presented in FIG. 1(ii).

Concomitant anti-itch therapy use was low, with antihistamines used most often (9-22%).

### Measurement of Level of Pharmacodynamic Biomarkers

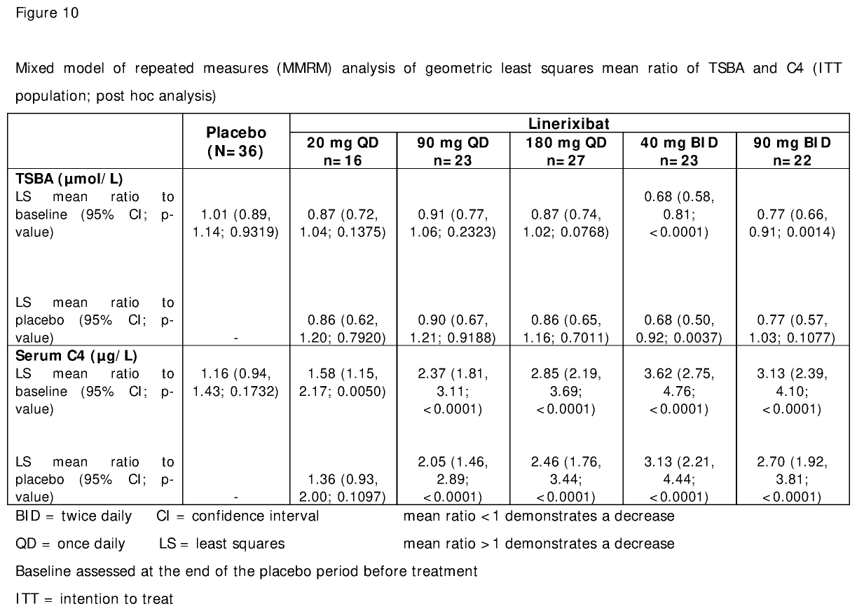

Total serum bile acids (TSBA) (FIG. 10)

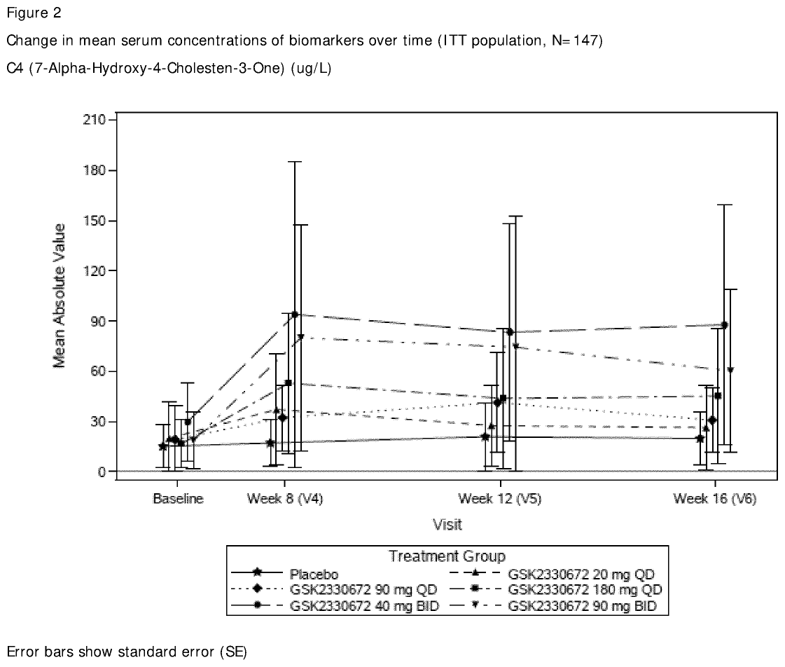

Serum 7-α-hydroxy-4-cholesten-3-one (C4) (FIG. 2)

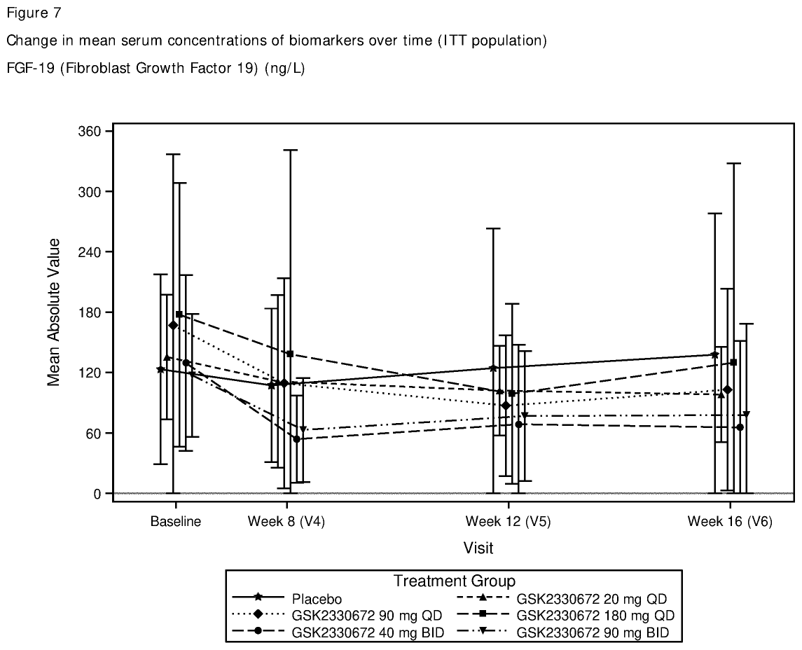

Serum fibroblast growth factor-19 (FGF-19) (FIG. 7)

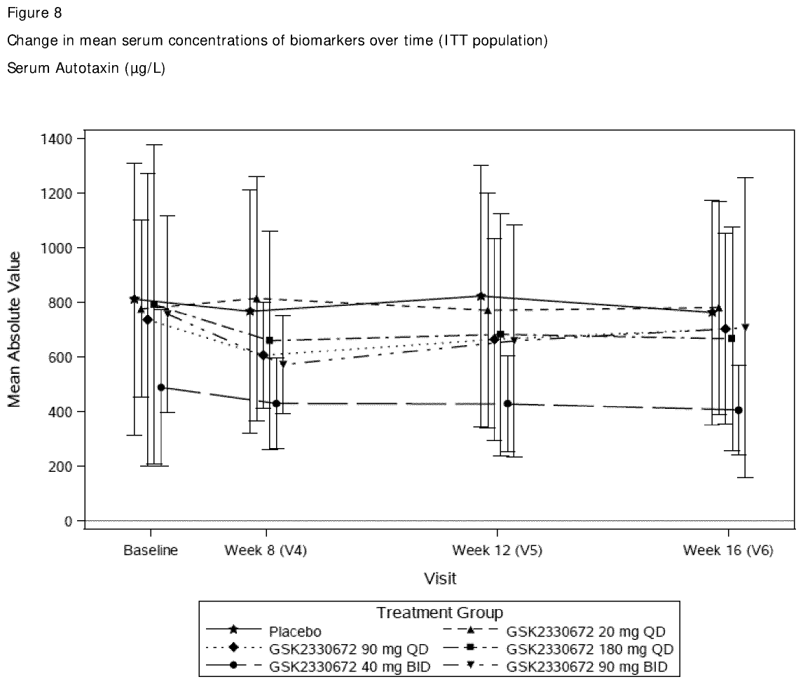

Serum autotaxin (FIG. 8)

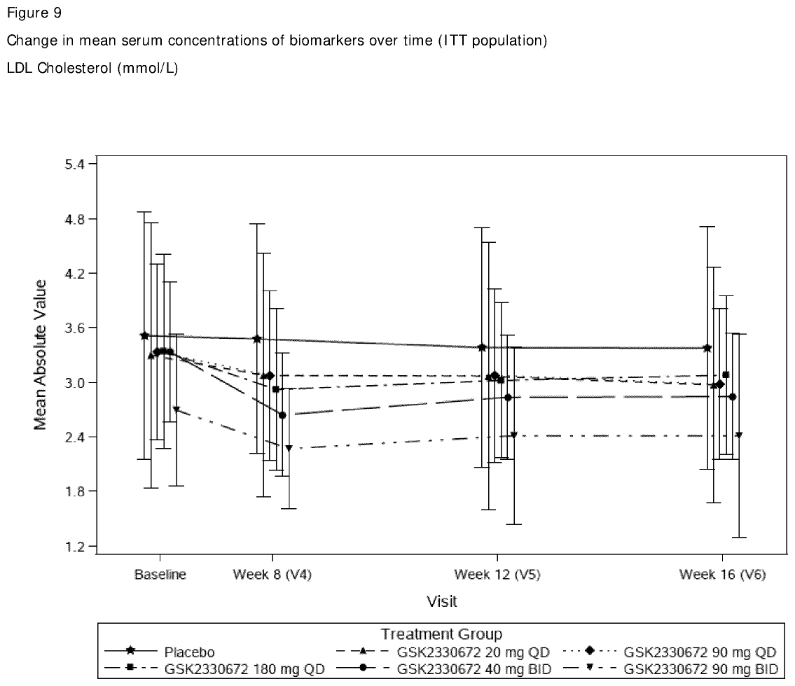

Serum LDL (low-density lipoprotein) cholesterol (FIG. 9)

A rapid, treatment-related change in the biomarkers, including TSBA, C4, FGF-19, serum autotaxin and LDL cholesterol, was evident at Week 8 and was maintained during treatment. The largest increase in C4 was apparent with the linerixibat 40 mg BID and 90 mg BID doses (FIG. 2). The two BID doses of 40 mg and 90 mg resulted in the greatest reduction in 10 fibroblast growth factor-19 (FGF-19), shown in FIG. 7.

Serum autotaxin concentrations decreased across treatment groups and at Week 16 was significantly decreased for the 40 mg BID group compared with placebo consistent with a purported role in itch.

Linerixibat inhibition of IBAT results in increased cholesterol synthesis to bile acids with C4 and increased expression of the LDL receptor, reducing circulating cholesterol (FIG. 9). With the exception of the 20 mg QD group, all linerixibat treatment groups significantly reduced LDL cholesterol at Week 16 compared to the placebo group.

Further analysis of biomarker parameters provided clear indication of pharmacological action with decreases in total serum bile acids (TSBA), increases in the serum bile acid precursor C4 (7-a-hydroxy-4-cholesten-3-one) and corresponding mean decreases in fibroblast growth factor 19 (FGF-19), a hormone that regulates bile acid synthesis. A mixed model repeated measures (MMRM) analysis (FIG. 10) demonstrated a statistically significant decrease in total serum bile acids (TSBA) at week 16 for the linerixibat 40 mg and 90 mg BID treatment groups compared with baseline and a significant difference for the 40 mg BID treatment group compared with placebo. In addition, MMRM analysis of C4 showed that the largest increases from baseline of C4 (“least squares” (LS) mean ratio increases of C4) were observed in the linerixibat 180 mg QD and both BID treatment groups. Reductions in FGF-19 and autotaxin were also observed across most treatment groups during the main study period (excluding placebo for FGF-19 and linerixibat 20 mg QD for autotaxin) (FIG. 7, FIG. 8). The Analysis of covariance (ANCOVA) of mean change from baseline for FGF-19 showed statistically significant reductions for linerixibat 40 mg BID compared with placebo.

### Efficacy

### Itch Relief

The pre-specified primary endpoint of the study was not met in that a dose response could not be established across dose ranges for both the ITT and per protocol populations.

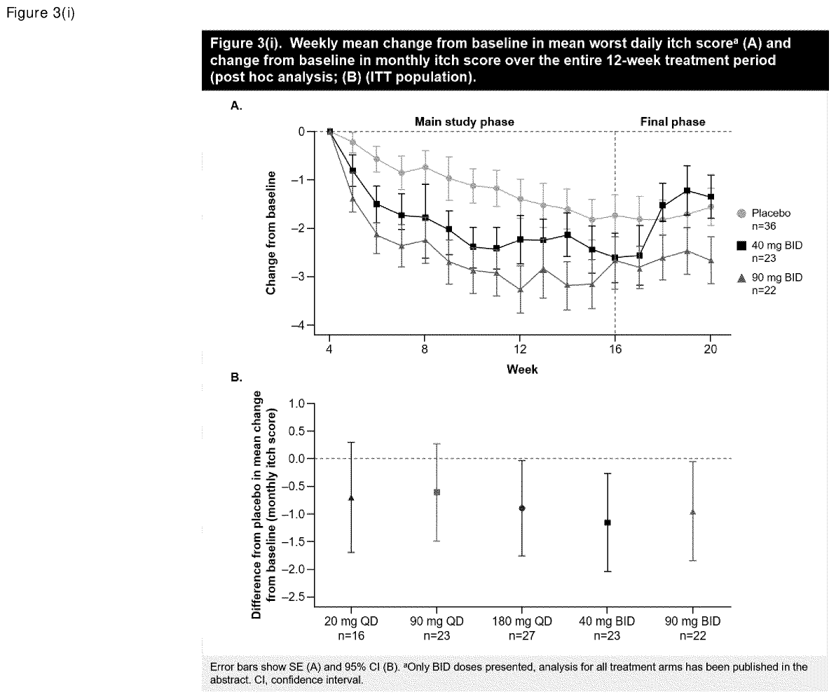

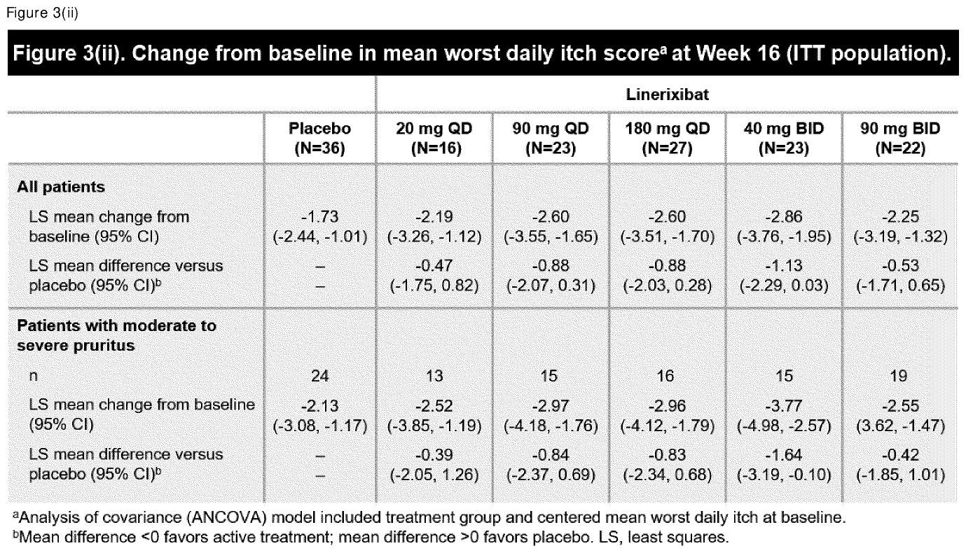

All linerixibat doses showed rapid improvement in mean Worst Daily Itch Score, with worsening itch after treatment withdrawal. FIG. 3(i) part A shows BID doses and placebo only. However, there was a high placebo response (FIG. 3(ii)).

Due to the potential fluctuation of itch over time, an additional post-hoc analysis was conducted on the primary endpoint according to the FDA preferred approach to evaluate the effect over the entire 12 weeks treatment duration using Monthly Itch Score. A mixed model repeated measures (or mixed-effect repeated measure model) (MMRM) analysis estimated the effect of linerixibat comparing to placebo over the entire 12 weeks treatment duration. Three active doses demonstrated a statistically significant reduction relative to placebo in change from baseline in the Monthly Itch Score over 12 weeks treatment ((FIG. 3(i), B) [Least Squares (LS) Mean Change from Baseline vs Placebo: 40 mg BID -1.16 (p=0.011)), 90 mg BID −0.95 (p=0.037), and 180 mg QD −0.90 (p=0.042)]

Mean Worst Daily Itch Score of patients with moderate to severe itch at baseline showed a significant difference for linerixibat 40 mg BID versus placebo (FIG. 3(ii)).

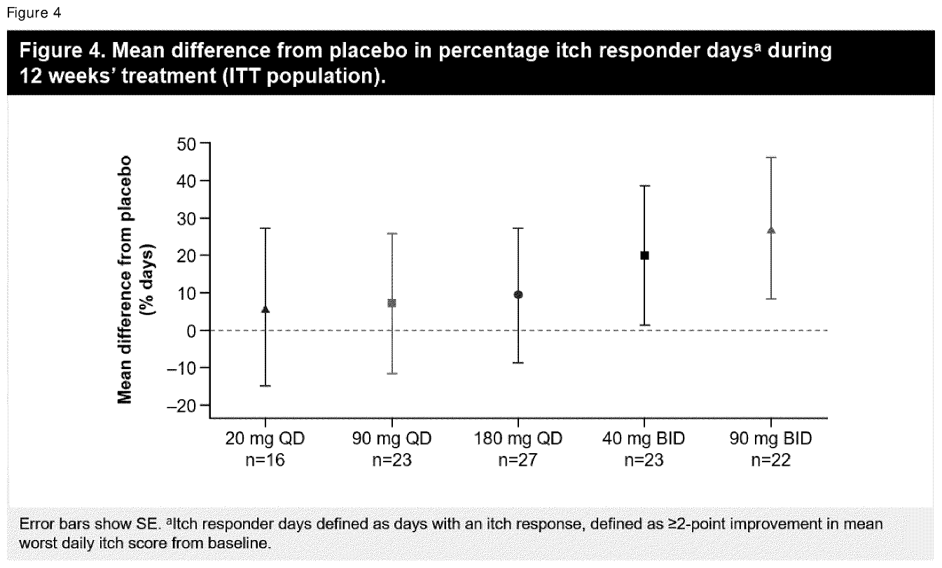

The linerixibat 40 mg BID and 90 mg BID groups showed a respectively 20% and 27% mean increase in itch responder days (FIG. 4).

### PBC-40

All treatment groups including placebo showed a significant improvement from baseline in the PBC-40 itch domain after 12 weeks (p≤0.05).

The 40 mg BID group showed significant improvement in PBC-40 social and emotional domains from baseline (LS mean difference: −3.1 (95% CI: −5.1, −1.2)) (LS Mean difference vs placebo −2.4[95% CI:−4.8, 0.1]) and −1.4 (95% CI: −2.2, −0.5) (LS Mean difference vs placebo −0.8 [95% CI: −1.9, 0.3]), respectively.

### Conclusions

Biomarkers and dose response data favour BID linerixibat dosing.

12 weeks' linerixibat treatment showed evidence of rapid and significant improvement in itch in patients with PBC and cholestatic pruritus.

Significant difference versus placebo following 12 weeks treatment in moderate to severe patients (40 mg BID).

Significant improvement from baseline over 12 weeks treatment in Monthly Itch Score (40 mg and 90 mg BID; 180 mg QD).

Significantly more responder days for 40 mg BID and 90 mg BID for linerixibat vs placebo.

Significant improvement from baseline in PBC-40 social and emotional domains with 40 mg BID.

Most common AE was diarrhea, as anticipated due to the treatment's mode of action.

Targeting bile acid reuptake with linerixibat may provide relief for patients with PBC and cholestatic pruritus.

### Analysis of Relationship Between Itch Severity and Sleep Interference

In this study, the impact of linerixibat on itch in patients with PBC was further evaluated. The analysis explores the relationship between Itch Severity and sleep interference. The downstream impact of pruritus on sleep was measured using a sleep interference NRS. The impact of pruritus on health-related quality of life (HRQOL) was measured using the PBC-40.

### Measurement of Sleep

Daily Sleep Score: a sleep interference score recorded by study participants once daily in the morning using a 0-10 numeric rating scale (NRS), where 0 represents no sleep interference and 10 represents complete sleep interference

Mean Daily Sleep Score (Weekly Sleep Score [WSS]): the Daily Sleep Scores are averaged over 7 days.

Monthly Sleep Score (MSS): the worst Mean Daily Sleep Score for that month (i.e., worst week score of the 4 weeks).

### Background and Aims

As indicated above, pruritus is a common symptom in primary biliary cholangitis (PBC). It can affect sleep; this may impair quality of life, but objective data are limited. The present analysis explores the relationship between Itch Severity and sleep interference.

### Methods

Patients in the GLIMMER trial recorded itch (twice daily) and degree of sleep interference from itch (each morning) on a 0-10 numeric rating scale. Worst Daily Itch Scores and sleep interference scores and Worst Daily Itch Scores were averaged over 1 week to generate mean scores (weekly Itch Score [WIS] and weekly sleep score [WSS]). Monthly sleep/itch scores were based on the most severe WIS and WSS scores in a given month (MIS and MSS, respectively), i.e. worst weekly score for that month. MIS and MSS calculations and analyses were post hoc as was the correlation of MIS versus MSS (Months 1-3) using Bland—Altman repeated measures analysis. Additional analyses consisted of prespecified exploratory psychometric evaluations between WIS, WSS and other patient-reported outcome (PRO) measures used in the study which were conducted as part of the patient reported outcome psychometric validation, in particular, the 5-D itch measure, which was assessed at initial study period (Day 1), baseline (Day 28), Week 12 of treatment (Day 112) and final study period (Day 140).

### Results

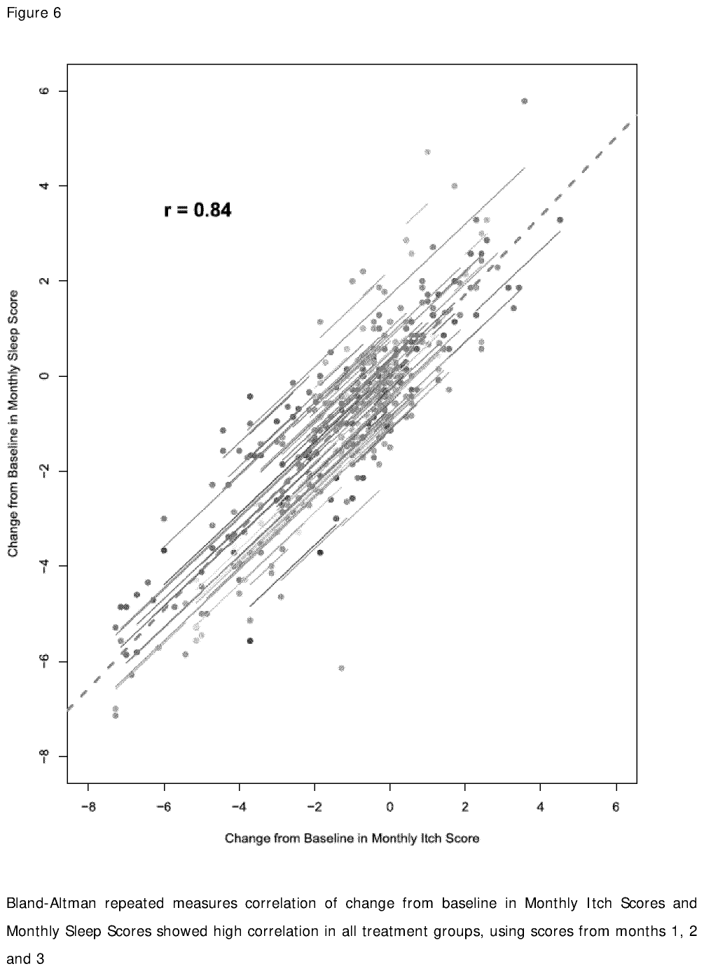

The intent-to-treat (ITT) population comprised 147 patients. All analyses were conducted on the ITT population. Improvements in change in MIS and MSS from baseline were highly and significantly correlated (r=0.84; p<0.0001; FIG. 6). See FIG. 6 for a Bland-Altman graph of the correlation of change from baseline between Monthly Sleep Score and Monthly Itch Score. Convergent validity analyses from the psychometric validation analyses of WSS at baseline showed moderate correlations with the 5-D itch total (r=0.59; p<0.0001) and 5-D itch sleep item scores (r=0.58; p<0.0001).

### Conclusions

Itch symptoms closely correlated with sleep interference, suggesting the potential for a possible close clinical relationship between itch and sleep; the correlation between 5-D itch total and WSS support this. In addition to improving the direct impact of the symptom of itch, an improvement in itch may have an impact on sleep. This is of importance, as sleep disturbance is a common complaint of patients with PBC that adds to disease burden.

### Discussion

In the GLIMMER trial, once daily dosing (QD) was expected to be the optimum dosing regimen. The rationale for QD was as follows: i) it was considered optimal to administer one dose in the morning alone, avoiding administering a dose at night in order to minimise the known side effect of diarrhoea during the night, and ii) one dose per day is generally expected to result in a better patient compliance. The tablet sizes employed in the trial were 10 mg and 45 mg which allowed administration of different dose sizes by combining tablets.

As illustrated in FIG. 1(i), the initial dose sizes were: placebo, 20 mg QD, 90 mg QD and 180 mg QD. Also included was a dose size of 90 mg BID which was evaluated in the earlier Phase 2a study. This study had a prespecified adaptive design and interim analysis. A detailed interim analysis of the safety and dose response data indicated that there was an increased effect at a higher dose and that surprisingly, 90 mg BID appeared to be better tolerated than 180 mg QD. This warranted a further investigation of twice daily dosing. It was decided to add a new 40 mg BID group, and to cease the 20 mg QD dose.

TABLE 1

| | |
|---|---|
| Protocol: 201000 | Page 5 of 12 |
| Population: Safety | |

Table 3.1
Summary of All Adverse Events by Study phase

| | System Organ Class | Placebo | | 20 mg QD | | 90 mg QD | | 180 mg QD | | 90 mg BID | |
|---|---|---|---|---|---|---|---|---|---|---|---|
| Study phase | Preferred Term | (N = 22) | | (N = 14) | | (N = 12) | | (N = 17) | | (N = 13) | |
| Main Study | ANY EVENT | 11 | (50%) | 8 | (57%) | 9 | (75%) | 14 | (82%) | 9 | (69%) |
| | Uncoded | | | | | | | | | | |
| | Any event* | — | | — | | — | | — | | — | |
| | Uncoded* | — | | — | | — | | — | | — | |
| | Gastrointestinal disorders | | | | | | | | | | |
| | Any event | 5 | (23%) | 7 | (50%) | 6 | (50%) | 14 | (82%) | 5 | (38%) |
| | Diarrhoea | 2 | (9%) | 5 | (36%) | 6 | (50%) | 10 | (59%) | 5 | (38%) |
| | Abdominal pain | 2 | (9%) | 2 | (14%) | 1 | (8%) | 5 | (29%) | 1 | (8%) |
| | Nausea | 1 | (5%) | 1 | (7%) | 0 | | 1 | (6%) | 2 | (15%) |
| | Vomiting | 0 | | 1 | (7%) | 0 | | 2 | (12%) | 1 | (8%) |
| | Abdominal pain upper | 0 | | 2 | (14%) | | | 1 | (6%) | 0 | |
| | Dry mouth | 0 | | 2 | (14%) | 1 | (8%) | 0 | | 0 | |
| | Abdominal distension | 0 | | 0 | | 1 | (8%) | 1 | (6%) | 0 | |
| | Abdominal pain lower | 0 | | 1 | (7%) | 1 | (8%) | 0 | | | |
| | Flatulence | 0 | | 1 | (7%) | 0 | | 1 | (6%) | 0 | |
| | Gastrooesophageal reflux disease | 1 | (5%) | 1 | (7%) | 0 | | 0 | | 0 | |

Table 1 shows 5 out of 13 (38%) patients reported gastrointestinal adverse events (GI AEs) in the 90 mg BID group vs 14 out of 17 (82%) patients in the180 mg QD group. Diarrhea and abdominal pain were of particular interest:

Diarrhea is 38% 90mg BID group vs 59% 180 mg QD group

Abdominal pain is 90 mg BID group 8% vs 29% 180 mg QD group (noting the 180 mg QD group also had one adverse event each of abdominal pain upper, abdominal distension and flatulence)

At the end of the study overall, compared to placebo, the 40 mg BID dose demonstrated significant improvement in pruritus over 12 weeks of treatment in the ITT population, significant improvement in pruritus following 12 weeks of treatment in participants with moderate to severe itch at baseline, showed significant mean number of responder days 2 point improvement in mean worst daily itch) as well as significant change from baseline in the social and emotional domains of PBC-40. Gastrointestinal tolerability data also favored 40 mg BID dosing over higher doses.

1-13. (canceled)

14. A method of treatment of cholestatic pruritus in primary biliary cholangitis in a human in need thereof comprising administering to said human the compound 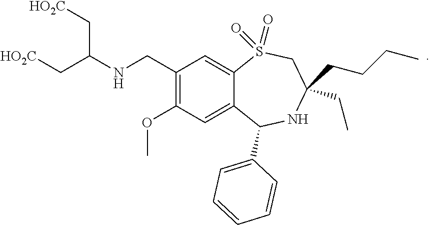 [SMILES: CCCC[C@]1(CC)CS(=O)(=O)c2cc(CNC(CC(=O)O)CC(=O)O)c(OC)cc2[C@@H](c2ccccc2)N1]
or a pharmaceutically acceptable salt thereof, wherein the compound is administered in an amount of approximately 40 mg, twice daily.

15. A method of treatment according to claim 14, wherein such administration results in improvement in itch.

16. The method of treatment according to claim 14 wherein the compound is administered orally.

17. The method of treatment according to claim 14 wherein the compound is:

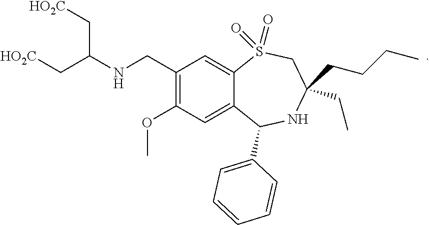 [SMILES: CCCC[C@]1(CC)CS(=O)(=O)c2cc(CNC(CC(=O)O)CC(=O)O)c(OC)cc2[C@@H](c2ccccc2)N1]

18. The method of treatment according to claim 14 wherein said improvement in itch is demonstrated by a mean difference from placebo in mean change from baseline Monthly Itch Score of at least −0.5.

19. The method of treatment according to claim 14 wherein said improvement in itch is demonstrated by a mean difference from placebo in mean change from baseline Monthly Itch Score of at least −0.9.

20. The method of treatment according to claim 14 wherein the treatment period is at least 12 weeks in duration.

21. A method of treatment of cholestatic pruritus in primary biliary cholangitis in a human in need thereof comprising administering to said human the compound 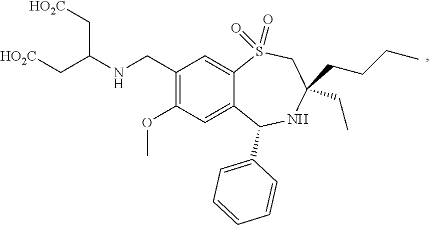 [SMILES: CCCC[C@]1(CC)CS(=O)(=O)c2cc(CNC(CC(=O)O)CC(=O)O)c(OC)cc2[C@@H](c2ccccc2)N1]
or a pharmaceutically acceptable salt thereof, wherein the compound is administered twice daily, and wherein such administration results in improvement in itch.
